(19) United States
(12) Patent Application Publication
(10) Pub. No.: US 2017/0333677 A1
(43) Pub. Date: Nov. 23, 2017

(54) Dialysis Catheter Insertion Assembly and Method

(71) Applicant: VascuTech Medical LLC, Oaks, PA (US)

(72) Inventor: Mahase Nardeo, Oaks, PA (US)

(73) Assignee: VascuTech Medical LLC, Oaks, PA (US)

(21) Appl. No.: 15/581,181

(22) Filed: Apr. 28, 2017

Related U.S. Application Data
(60) Provisional application No. 62/340,000, filed on May 23, 2016.

(51) Int. Cl. A61M 25/01; A61M 25/09; A61B 34/20; A61M 39/10; A61M 25/00

(52) U.S. Cl. CPC A61M 25/0102; A61M 39/10; A61M 25/003; A61B 34/20; A61M 25/09; A61M 2025/0031; A61M 2025/0037

(57) ABSTRACT

Disclosed is a catheter insertion assembly that includes an arterial lumen having a first stylet embedded therein and a venous lumen having a second stylet embedded therein, The stylets extend beyond the proximal and distal ends of each of the lumens. The second stylet of the venous lumen includes an aperture near the distal end thereof so as to receive the distal end of the first stylet of the arterial lumen therethrough, thereby connecting the venous and arterial lumens temporarily together when the catheter is being inserted into a patient. In this way, the stylets increase column strength for dual-tip catheters.

### CROSS-REFERENCE TO RELATED APPLICATIONS

This application claims the benefit of U.S. Provisional Patent Application No. 62/340,000, filed May 23, 2016, which is hereby incorporated by reference in its entirety.

### FIELD OF THE INVENTION

The present invention relates generally to medical devices, namely, catheters. More particularly, the present invention is directed to a catheter insertion assembly for dual-tip catheters.

### BACKGROUND OF THE INVENTION

Dialysis catheters are generally inserted into the blood vessels using two different techniques: percutaneously (i.e., over a guide wire), and using a tearaway sheath. Catheters with a hard durometer can be easily inserted percutaneously because they have excellent column strength and do not kink easily during the insertion procedure. However, catheter with soft durometers cannot be easily inserted unless a stylet is fitted into the lumens to strengthen the create column.

Additionally, it is even more difficult to insert a catheter with a split tip because both lumens are separated and need to be properly attached together in order to be inserted easily. In this regard, the invention described herein addresses these problems.

### SUMMARY OF THE INVENTION

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as to prelude to the more detailed description that is disclosed later.

Some embodiments of the present invention comprise a venous lumen that is in a side-by-side configuration with an arterial lumen, wherein the two lumens are held together via a catheter hub. Each of the lumens comprises a stylet therein, further wherein the stylets extend beyond the proximal and distal ends of each of the lumens. The stylet for the venous lumen comprises an aperture near the distal end thereof so as to receive the distal end of the stylet for the arterial lumen therethrough, thereby connecting the venous and arterial lumens together. In this way, the stylets are configured to create column strength for dual-tip catheters such as split tip and step tip catheters.

It is, therefore, an objective of the present invention to provide a new and improved catheter insertion assembly for use with catheters with separated distal ends or dual-tips (e.g., split tip and step tip catheters).

It is another objective of the present invention to provide a new and improved catheter insertion assembly that increases the ease of inserting dialysis catheter with soft durometers.

It is still another objective of the present invention to provide a new and improved catheter insertion assembly that can temporarily attach the distal ends of split tip or step tip catheter together in order for the catheter to be inserted easily into the body.

It is still yet another objective of the present invention to provide a new and improved catheter insertion assembly that can properly attach the distal ends of split tip or step tip catheter together without requiring an additional fastening mechanism.

A final objective of the present invention to provide a new and improved catheter insertion assembly that can be retroactively installed in existing split tip or step tip catheters.

In the light of the foregoing, these and other objectives are accomplished in accordance with the principles of the present invention, wherein the novelty of the present invention will become apparent from the following detailed description and appended claims.

### BRIEF DESCRIPTION OF THE DRAWINGS

The above and other objects and advantages of the present invention will be apparent upon consideration of the following detailed description, taken in conjunction with the accompanying exemplary drawings, in which like reference characters refer to like parts throughout, and in which:

FIG. 1 depicts a close-up side cross-sectional view of an exemplary embodiment of the catheter insertion assembly of the present invention.

FIG. 2 depicts a close-up top-down view of the exemplary embodiment of the catheter insertion assembly.

FIG. 3 shows an overall side view of the exemplary embodiment of the catheter insertion assembly.

FIG. 4 shows a close-up cross-sectional view of the distal end of the catheter insertion assembly from the front.

### DETAILED DESCRIPTION OF THE INVENTION

The present invention is directed towards a catheter insertion assembly. For purposes of clarity, and not by way of limitation, illustrative views of the present invention are described with references made to the above-identified figures. Various modifications obvious to one skilled in the art are deemed to be within the spirit and scope of the present invention.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to disclose concepts in a concrete fashion. As used in this application, the term “or” is intended to mean an inclusive “or” rather than an exclusive “or.”

Additionally, the articles “a” and “an” as used in this application and the appended claims should generally be construed to mean “one or more” or “at least one” unless specified otherwise or clear from context to be directed to a singular form. Similarly, the terms “plurality” and “a plurality” as used herein includes, for example, “multiple” or “two or more.” For example, “a plurality of items” includes two or more items.

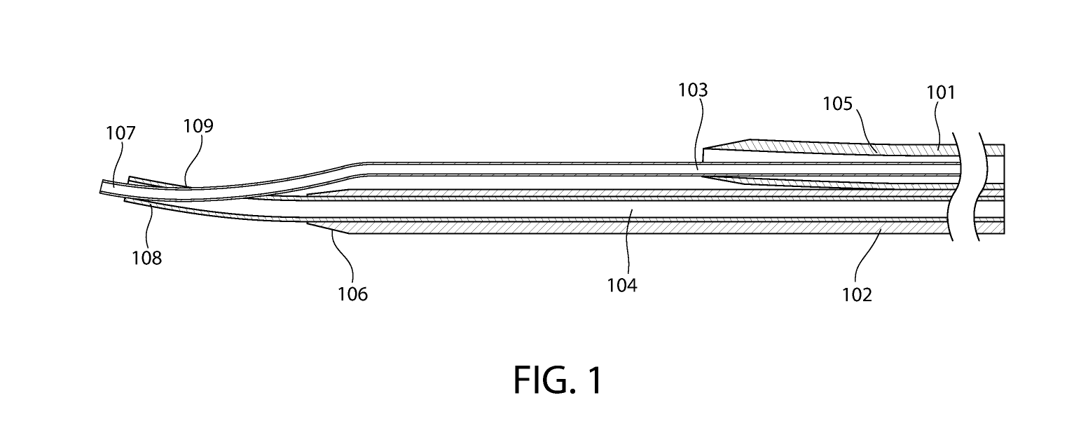

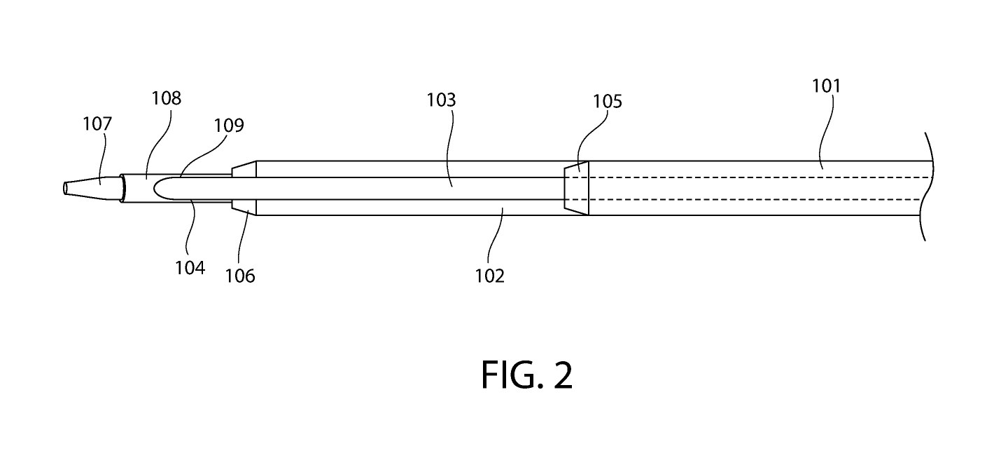

Referring now to FIGS. 1 and 2, there is shown a cross-sectional side view and a top-down view of the catheter insertion assembly, respectively. The catheter insertion assembly comprises a catheter having an arterial lumen 101 and a venous lumen 102, wherein each of the lumens 101, 102 comprises a substantially D-shaped cross section having a flat side opposite a rounded side. The flat side of the arterial lumen 101 can directly contact the flat side of the venous lumen 102 such that the arterial lumen 101 and the venous lumen 102 are in a side-by-side configuration to form a substantially circular cross section.

Each of the lumens 101, 102 comprises an open distal end 105, 106 and an open proximal end, wherein each of the distal end 105, 106 is tapered such that the diameter of the cross-section of the distal end 105, 106 is less than the diameter of the cross-section of the proximal end. The distance between the distal end 105 and the proximal end of the arterial lumen 101 defines the length of the arterial lumen 101. Similarly, the distance between the distal end 106 and the proximal end of the venous lumen 102 defines its length. The length of the arterial lumen 101 is less than the length of the venous lumen 102.

Preferably, the catheter comprises a split tip (as depicted in FIG. 1) or step tip catheter (as depicted in FIG. 2). The split tip catheter comprises lumens having distal ends that are apart and separate. The step tip catheter comprises a venous lumen having a distal end that extends beyond a distal end of an arterial lumen. In both embodiments, the venous lumen 102 and the arterial lumen 101 can comprise separate lumens. Said another way, each of the lumens comprises a separate structure and are bonded or held together, for example, via a sleeve.

Alternatively, the lumens may be substantially unitary in structure and split or separated in half via a septum to define two separate lumens—a venous lumen and an arterial lumen. In some embodiments, the catheter can comprise other types of catheters, including catheters comprising lumens with high durometer or lumens that tend to easily bend or flex when being inserted.

Each of the lumens 101, 102 further comprises a stylet embedded therein. It is noted that the term “stylet” or “stylets” may be used interchangeably with the terms “mandrel” or “mandrels.” In the illustrated embodiment, the arterial lumen 101 comprises a first stylet 103 therein. Similarly, the venous lumen 102 comprises a second stylet 104 therein. The stylets 103, 104 can be shaped and dimensioned to fit within the interior of the lumens 101, 102.

Accordingly, some embodiments of the first and second stylets 103, 104 also comprises a substantially D-shaped cross section. Each of the first and second stylets 103, 104 comprises an open proximal end and an open distal end 107, 108. The first stylet 103 comprises a tapered distal end 107. In the illustrated embodiments, the distal end 107 of the first stylet 103 comprises a substantially round or circular cross section. Preferably, the distal end 107 of the first stylet 103 is configured to fit over a guide wire.

The first stylet 103 comprises a sufficient length such that the distal end 107 of the first stylet 103 extends beyond the distal end 105 of the arterial lumen 101, and the proximal end of the first stylet 103 extends beyond the proximal end of the arterial lumen 101. Similarly, the second stylet 104 also comprises a sufficient length so that the distal end 108 of the second stylet 104 extends beyond the distal end 106 of the venous lumen 102, and the proximal end of the second stylet 104 extends beyond the proximal end of the venous lumen 102.

The distal end 108 of the second stylet 104 comprises an aperture or an opening 109 at an angle so as to receive the distal end 107 of the first stylet 103 therethrough. Because the aperture 109 is drilled at an angle relative to the axis perpendicular to the length of the second stylet 104, the aperture is substantially oblique or oval in shape instead of a circle. In one embodiment, the aperture 109 is disposed approximately 5 mm from the distal end 108 of the second stylet 104. It is contemplated, however, that the exact location and shape of the aperture 109 can vary depending upon embodiments.

In operation, the distal end 107 of the first stylet 103 is inserted through the aperture 109 and exited through the open distal end 108 of the stylet 104 so as to keep the two lumens 101, 102 together when being inserted. Preferably, the distal end 107 of the first stylet 103 extends slightly beyond the distal end 108 of the second stylet 104. Thus, the diameter of the cross section of the tapered distal end 107 of the first stylet 103 is less than the diameter of the cross-section of the distal end 108 of the second stylet 104. It is noted, however, that the first stylet 103 fills the interior of the second stylet 104 to fill the entire arterial and venous lumen so that there is no gap for tissue to get caught within the gap.

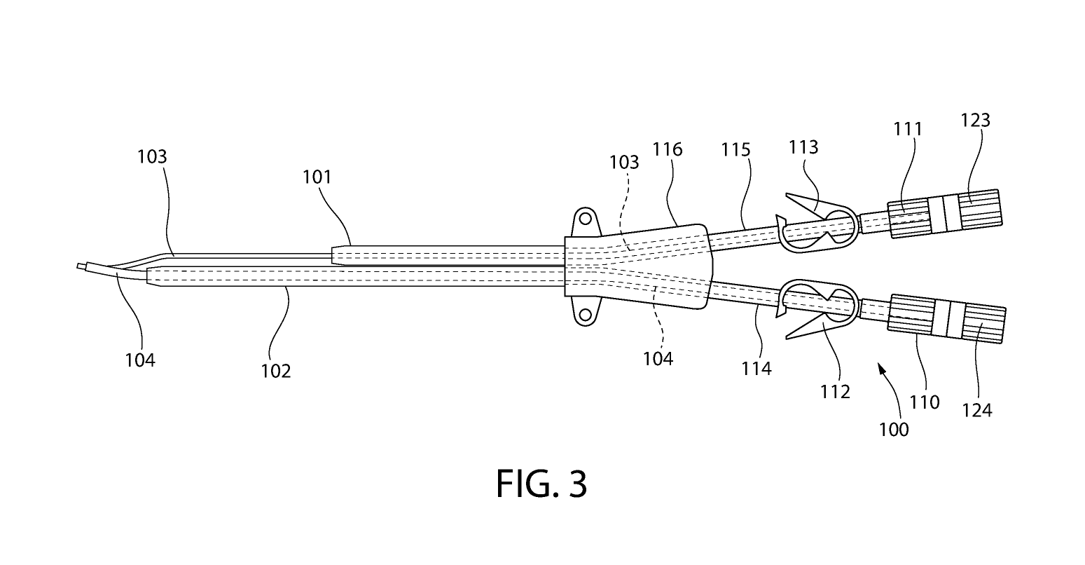

Referring now to FIG. 3, there is shown an overall side view of the catheter insertion assembly 100. The proximal ends of the lumens 101, 102 are connected to the distal ends of a venous extension 115 and an arterial extension 114, respectively, via an insert molding process, and attached at a catheter hub 116.

In some embodiments, the catheter 100 further comprises a sleeve or a cap for holding the lumens 101, 102 together, wherein the sleeve is biased toward the hub 116 so as to leave the distal ends of the lumens 101, 102 free. It is contemplated, however, that the sleeve can travel along the length of the lumens 101, 102 before being secured to the hub 116. In some embodiments, the sleeve comprises interior threads that mate with exterior threads disposed on the hub 116 so as to secure the sleeve in place.

The venous extension 115 and the arterial extension 114 extend through the hub 116. The proximal end of the venous extension 115 is connected to a first catheter female luer 111, and the proximal end of the arterial extension 114 is connected to a second catheter female luer 110. The luers 111, 110 are configured to connect to blood lines. In some embodiments, the venous extension 115 and the arterial extension 114 further comprise clamps 113, 112 thereon.

The first and second stylets 103, 104 extend through the venous extension 115 and the arterial extension 114 such that the proximal ends of the stylets 103, 104 exit the luers 111, 110. The proximal ends of each of the stylets 103, 104 can comprise a male luer fitted with a spin lock connector 123, 124. The male luer 123 at the proximal end of the first stylet 103 is attached to a first catheter female luer 111 via a spin connector and the male luer 124 at the proximal end of the second stylet 104 is attached to a second catheter female luer 110 via another spin connector. Each of the male luers at the proximal ends of the stylets 103, 104 can be attached via insert molding or solvent molding, depending upon embodiments.

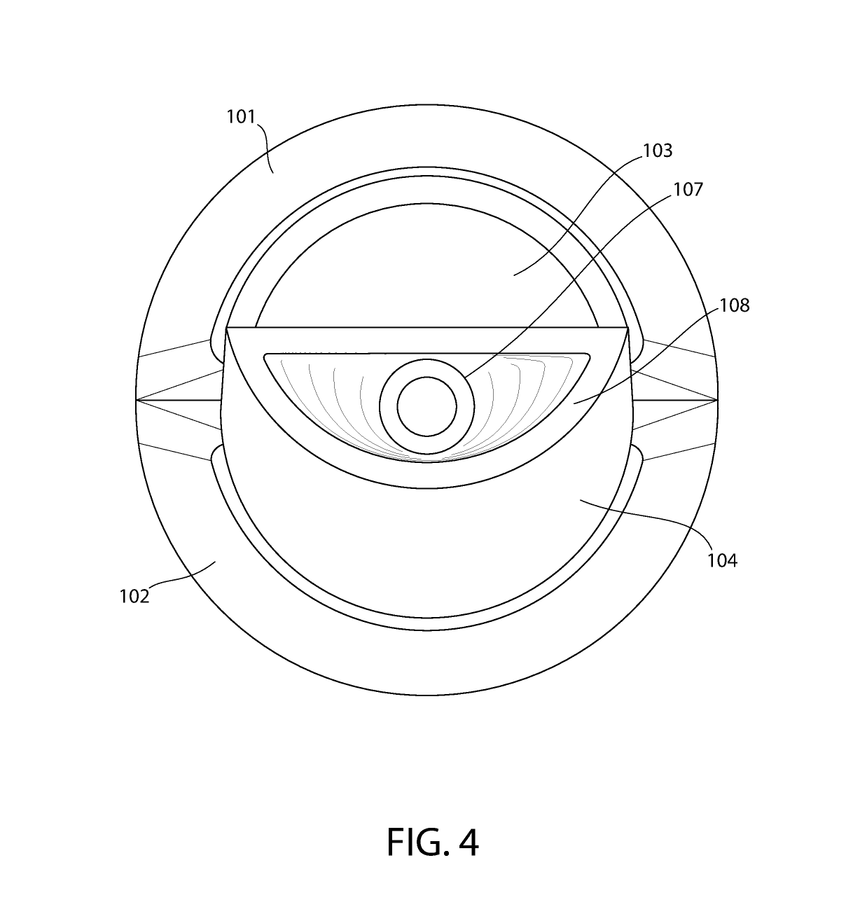

To temporarily secure the first and second stylets 103, 104 together as depicted in FIG. 4 and still allow for the insertion of a guide wire, each of the two stylets 103, 104 is embedded within or inserted through each of the respective lumens 101, 102. In operation, the desired blood vessel is accessed with a needle (e.g., an 18 G needle). A guide wire is inserted into the needle and pushed until the guide wire is positioned into place by using fluoroscopy guidance or a similar guidance method. A dilator is then inserted over the guide wire to dilate the blood vessel so that the catheter can be inserted with ease.

Once the dilator is removed, the distal end 107 of the first stylet 103 is inserted over the wire and gradually pushed into place by, for example, using fluoroscopy guidance. The guide wire is configured to exit the proximal extension of the arterial lumen 101. Once the catheter is in place, the guide wire is removed and the catheter is sutured in place. The stylets 103, 104 can be removed after the catheter is in the proper location by pulling on the back end of the first stylet 103 from the proximal end of the catheter, releasing it from the aperture 109 of the second stylet 104. Once the two stylets 103, 104 are separated, both stylets 103, 104 are removed from the lumens 101, 102.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A catheter insertion assembly, comprising:
a catheter having an arterial lumen and a venous lumen, each of said arterial lumen and said venous lumen comprising a distal end and a proximal end;
a first stylet embedded within said arterial lumen, wherein a distal end of said first stylet extends beyond said distal end of said arterial lumen;
a second stylet embedded said venous lumen, wherein a distal end of said second stylet extends beyond said distal end of said venous lumen, further wherein said distal end of said second stylet comprises an aperture configured to receive a distal end of said first stylet therethrough;
said distal end of said first stylet inserted through said aperture and exiting through said distal end of said second stylet, thereby maintaining said arterial lumen and said venous lumen temporarily together.

2. The catheter insertion assembly of claim 1, wherein said proximal end of each of said first stylet and said second stylet is attached to a luer.

3. The catheter insertion assembly of claim 2, wherein said luer is attached to each of said proximal end of said first stylet and said proximal end of said second stylet via insert molding or solvent molding.

4. The catheter insertion assembly of claim 1, wherein said distal end of said first stylet tapered.

5. The catheter insertion assembly of claim 1, wherein said catheter is a split tip catheter.

6. The catheter insertion assembly of claim 1, wherein said catheter is a step tip catheter.

7. The catheter insertion assembly of claim 1, further comprising:
a catheter hub connecting said proximal end of said arterial lumen to a distal end of an arterial extension and said proximal end of said venous lumen to a distal end of a venous extension;
a proximal end of each of said venous extension and said arterial extension connected to a luer.

8. A catheter insertion assembly, comprising:
a first stylet and a second stylet, each of said first stylet and said second stylet comprising an open proximal end and an open distal end;
said distal end of said second stylet comprising an aperture for receiving said distal end of said first stylet;
said distal end of said first stylet inserted through said aperture and exited through said distal end of said second stylet, thereby temporarily attaching said first stylet and said second stylet together.

9. The catheter insertion assembly of claim 8, wherein said second stylet is shaped and dimensioned to eliminate gap between said second stylet and an interior of said first stylet when said first stylet is inserted through said aperture of said second stylet and exited through said distal end of said second stylet.

10. The catheter insertion assembly of claim 8, wherein said distal end of said first stylet is tapered and comprises a circular cross section.

11. The catheter insertion assembly of claim 8, wherein said aperture is oblique or oval.

12. The catheter insertion assembly of claim 8, wherein said proximal end of each of said first stylet and said second stylet is attached to a luer.

13. The catheter insertion assembly of claim 8, wherein said first stylet is embedded in an arterial lumen and said second stylet is embedded in a venous lumen;
said distal end of said first stylet extending beyond a distal end of said arterial lumen;
said distal end of said second stylet extending beyond a distal end of said venous lumen.

14. The catheter insertion assembly of claim 13, wherein said distal end of said arterial lumen and said distal end of said venous lumen are split.

15. The catheter insertion assembly of claim 13, wherein said distal end of said arterial lumen and said distal end of said venous lumen are in a step configuration.

16. The catheter insertion assembly of claim 13, wherein said proximal end of each of said first stylet and said second stylet is attached to a luer.

17. A method of inserting a dual-tip catheter, comprising the steps of:
providing a catheter having an arterial lumen having a first stylet and a venous lumen having a second stylet, wherein a distal end of said first stylet is inserted through a distal end of said second stylet, thereby temporarily attaching said first stylet and said second stylet together;
inserting a guide wire into a blood vessel of a patient;
inserting said distal end of a first stylet over said guide wire;
removing said guide wire;
removing said distal end of said first stylet from said distal end of said second stylet;
removing said first stylet and said second stylet from said arterial lumen and said venous lumen.

18. The method of claim 17, wherein said distal end of said second stylet comprises an aperture for receiving said distal end of said first stylet therethrough.

19. The method of claim 17, wherein said catheter comprises a split tip catheter.

20. The method of claim 17, wherein said catheter comprises a step tip catheter.
